(19) United States
(12) Patent Application Publication
(10) Pub. No.: US 2006/0216171 A1
(43) Pub. Date: Sep. 28, 2006

(54) Hand held pump

(75) Inventor: Hector Ray Hernandez, Fullerton, CA (US)

Correspondence Address: John V. Hanley. Esq.;FULWIDER, PATTON, LEE & UTECHT, LLP, Howard Hughes Center, 6060 Center Drive, Tenth Floor, Los Angeles, CA, 90045, US

(73) Assignee: Alltrade Tools LLC

(21) Appl. No.: 11/088,956

(22) Filed: Mar. 23, 2005

(51) Int. Cl. A61M 1/00

(52) U.S. Cl. 417/437

(57) ABSTRACT

A hand held vacuum and pressure pump has a fixed handle, with a movable handle pivotally connected thereto at one end thereof, the fixed handle being fixed to a pump cylinder so as to be perpendicular thereto. A spring loaded piston rod extends from the movable handle through the fixed handle and is fixedly attached at one end to a piston which is contained in the pump cylinder and has a peripheral O-ring forming a seal therewith. Valve means controls whether manual movement of the movable handle toward the fixed handle forces fluid contained in the cylinder out of the cylinder or draws fluid into the cylinder. The movable handle has a longitudinal recess formed therein through which a connecting pin extends, the connecting pin being connected to the piston rod at the piston rod end remote from the piston, so that the arcuate movement of the movable handle toward and away from the cylinder permits the connecting pin to move longitudinally in the movable handle longitudinal recess, whereby the piston rod moves linearly when moving longitudinally in the cylinder, thereby avoiding canting the piston and so scuffing the O-ring when the piston rod moves longitudinally in the cylinder.

### BACKGROUND OF THE INVENTION

1. Field of the Invention

This invention relates to two-way manually operated hand-held pumps which are capable of selectively applying a vacuum or a pressure to the pump outlet.

2. Description of the Prior Art

Two-way manually operated hand-held pumps which are capable of selectively applying a vacuum or a pressure to the pump outlet, conventionally a nozzle type structure, are well known in the art. These pumps generally have a fixed handle and a movable handle and are classifiable into either of two types, depending upon the structural arrangement of their handles. Thus, U.S. Pat. No. 4,775,302, issued Oct. 4, 1988 to Theodore C. Neward, has a handle arrangement in the form of a Class 1 lever, whereas U.S. Pat. No. 6,558,130, issued May 6, 2003 to Hseuh Chin Chang, has a handle arrangement in the form of a Class 2 lever. As will be apparent to those skilled in the art, a Class 2 lever structure is more desirable from the point of view of requiring lesser manual strength to operate the pump, as both types depend upon the manual squeezing of a movable handle toward a fixed handle against a load or bias spring, which serves to return the movable handle to its rest position after the squeezing phase of the pump operation.

However, pumps of both types known in the prior art have suffered from a common deficiency, which arises out of the use of the pivotal lever principal for the handles. In pumps of both types, the pump has a piston which is fixed to a piston rod, the piston rod being pivotally connected to the movable handle. The piston is disposed in a pump cylinder and an O-ring on the periphery of the piston forms the required seal with the inner wall of the pump cylinder. However, the point of this pivotal connection between the piston rod and movable handle is offset longitudinally from the point of pivotal attachment between the two handles, so that the squeezing of the movable handle toward the fixed handle causes the movable handle, and so the piston rod, to move arcuately with respect to the fixed handle and so the pump cylinder. As it is not practical in this type of pump to connect the piston rod to the piston pivotally, as, for example, is the case for an internal combustion engine, because of cost and size considerations, the piston rod is conventionally fixedly attached to the piston. Because of the arcuate movement of the movable handle, and thus the piston rod and so the piston with respect to the pump cylinder interior wall, the piston cants within the cylinder during operation, rather than remaining perpendicular to the cylinder walls. This canting causes the O-ring which is typically used to seal the piston in the cylinder to tend to scuff, thus shortening the life of the O-ring, so as to require its replacement.

### SUMMARY OF THE INVENTION

According to the present invention, a hand held pump of the type which has a fixed handle, with a movable handle pivotally connected thereto so as to be arcuately movable with respect thereto, the fixed handle being fixed to a pump cylinder so as to be perpendicular thereto, the pump cylinder containing a piston with an O-ring forming a seal with the cylinder walls and with a piston rod fixed to the piston and pivotally connected to the movable handle, and a load spring disposed along at least a portion of the piston rod, whereby the pump is operated by selective relative movement between the movable handle and the fixed handle, has a longitudinal recess formed in the movable handle with a connecting pin extending through the longitudinal recess, and means connecting the connecting pin to the piston rod at the piston rod end remote from the piston, whereby, in response to arcuate movement of the movable handle toward and away from the cylinder during pump operation, the connecting pin moves longitudinally in the movable handle longitudinal recess, so that the piston rod moves linearly when moving longitudinally in the cylinder, thereby avoiding canting the piston in the cylinder and scuffing the O-ring during piston rod movement.

### BRIEF DESCRIPTION OF THE DRAWINGS

FIG. 1 is a left side elevational view, partially broken away, of a hand held vacuum and pressure pump according to the present invention when in its rest disposition;

FIG. 2 is a view, similar to FIG. 1, but with the pump in its partially actuated disposition;

FIG. 3 is a view, similar to FIGS. 1 and 2, but with the pump in its fully actuated disposition;

FIG. 4 is a partial fragmentary view in partial section, of the pump as shown in FIG. 3 in solid lines and in FIG. 1 in broken lines;

FIG. 5 is a partial sectional view taken along lines 5-5 of FIG. 1;

FIG. 6 is a partial sectional view taken along lines 6-6 of FIG. 1; and

FIG. 7 is a partial sectional view taken along lines 7-7 of FIG. 1.

### DETAILED DESCRIPTION OF THE PREFERRED EMBODIMENTS

Referring now to the drawings, a hand held vacuum and pressure pump 10 has a pump body 12 which includes a vacuum and pressure gage 14, a pump nozzle 16, a pump inlet-outlet control ring 18, and a valve assembly 19, all of which are conventional, and may be similar to those corresponding structures shown in U.S. Pat. Nos. 6,558,130 or 4,775,302, or other well known prior art devices, as will be readily apparent to those of ordinary skill in the art. The pump body 12 includes a cylindrical element 20 (hereinafter “cylinder 20”), within which a piston 22 is disposed. An O-ring 24 is positioned around the periphery of the piston 22 and forms a seal between the piston 22 and the walls of the cylinder 20.

The pump 10 has a fixed handle 26 and a movable handle 28, which are pivotally connected at one end of each thereof by a pivot pin 30 of conventional construction. The fixed handle 26 is fixed to the pump body 12 by any suitable conventional means, so that the fixed handle 26 is perpendicular to the cylinder 20. The cylinder 20 is closed by a cylinder plug 32, through which a piston rod 34 passes. The piston rod 34 is fixed to the piston 22 by any conventional means, such as being in threaded engagement therewith, or welded thereto, or in any other appropriate conventional manner so as to fixedly connect the two together. A load spring 36 is enclosed by a flexible boot 37 for cleanliness purposes, and is disposed between the fixed handle 26 and movable handle 28, and engages the movable handle 28 and the cylinder plug 32 so as to normally urge the movable handle 28 away from the fixed handle 26.

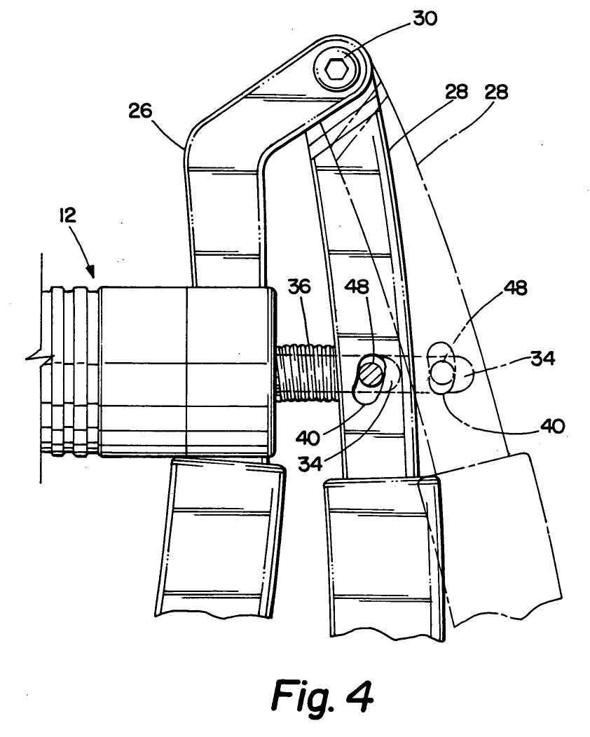

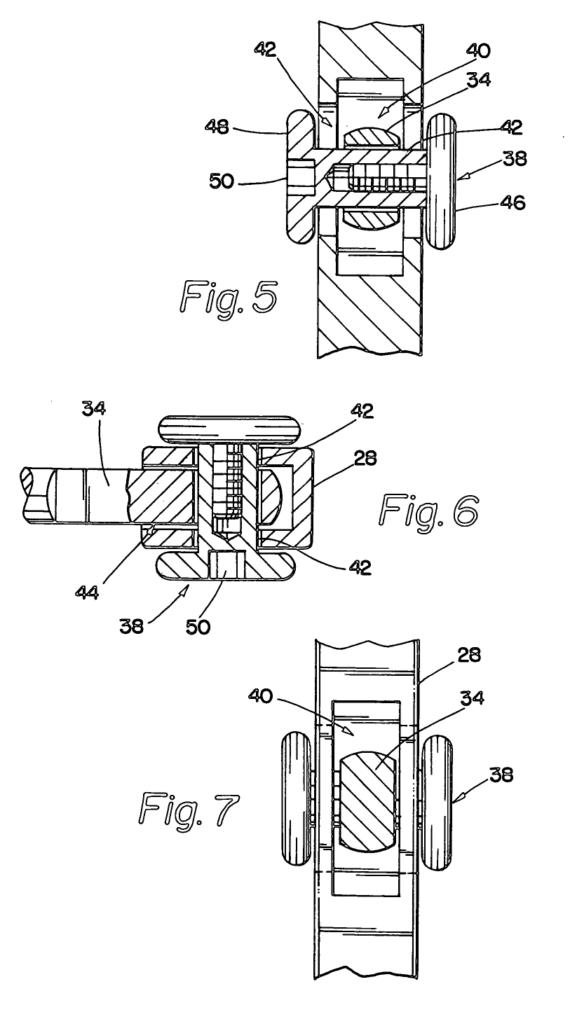

The piston rod 34, at its end remote from the piston 22, is connected to the movable handle 28 by means of a connecting pin 38, which extends through a longitudinal recess 40 formed so as to extend transversely through the movable handle 28, as is best shown in FIGS. 4 through 7. As is seen by comparing FIGS. 5 and 6, the longitudinal recess 40 is formed similar to a cross, with transverse arms 42 through which the connecting pin 38 extends, and an arm 44 through which the piston rod 34 extends. The connecting pin 38 itself is seen to be comprised of a first pin element 46, which is externally threaded, and a second pin element 40, which has a complementary internal thread, so that the two elements 38, 40, when threaded together, form a single connector which passes through the transverse arms 42 to connect the piston rod 34 to the longitudinal recess 40 of the movable handle 29, while permitting the piston arm 38 to move longitudinally within the movable handle 28, as the movable handle 28 moves toward or away from the fixed handle 26, in response to the manual actuation of the pump or the urging of the load spring 36, as appropriate. The second pin element 48 is shown in FIG. 5 as having a hex wrench-receiving recess 50, which may be used to tighten or loosen the attachment of the two pin elements 46, 48, if desired. If desired, the first pin element 46 has a similar recess 50 (see FIG. 6). Alternatively, the pin elements 46, 48 may have any other conventional configuration, such as polygonal peripheral surfaces, adapted to facilitate assembly and disassembly.

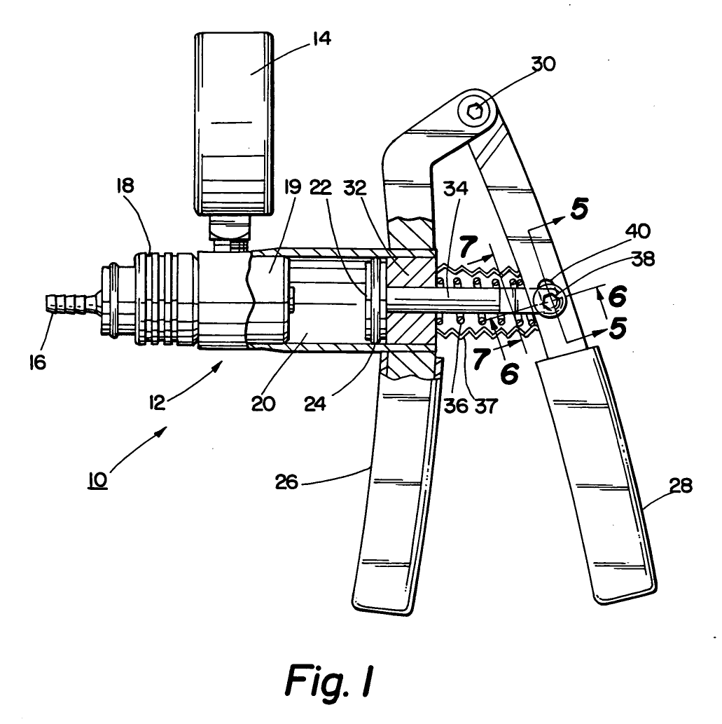

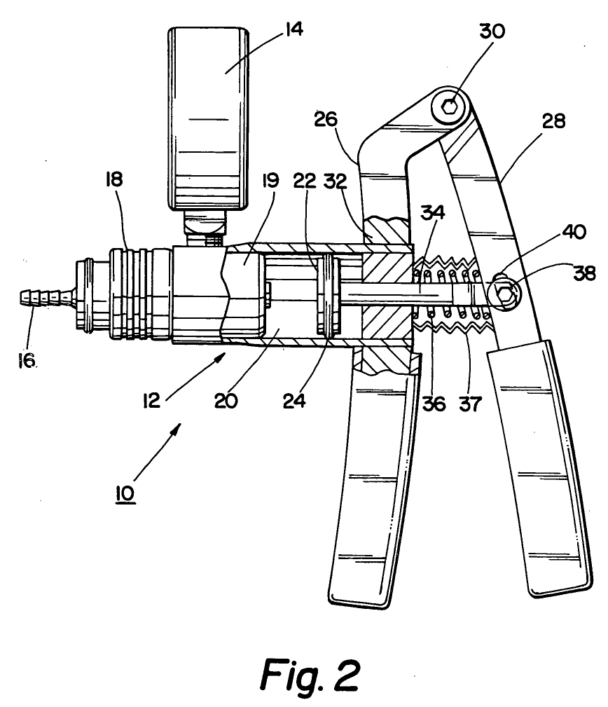

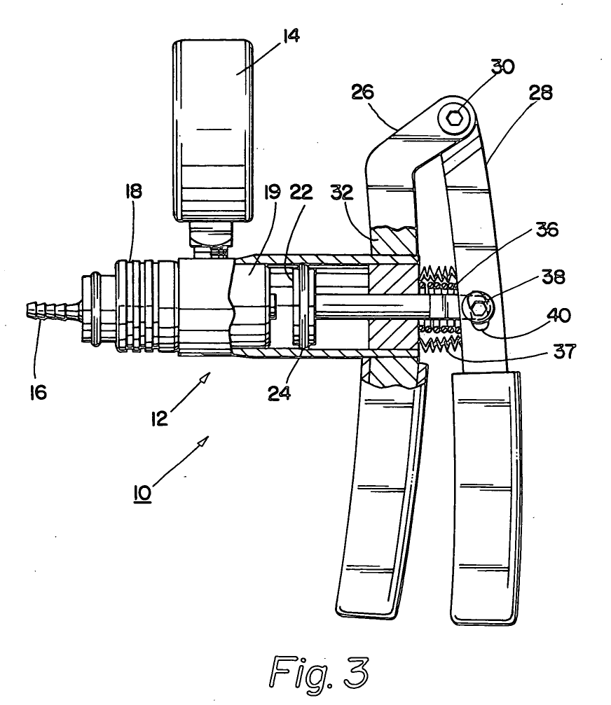

The operation of the pump 10 of the present invention is best illustrated by comparing FIGS. 1, 2 and 3, while FIG. 4 illustrates the detail of the movement of the connecting pin 38 in the longitudinal recess 40 during movement of the handles 26, 28 from the disposition shown in FIG. 1 to the disposition shown in FIG. 3. Thus, in operation, the load spring 36 is compressed from that disposition shown in FIG. 1 to the disposition shown in FIG. 3 by the manual squeezing together of the handles 26, 28. When the squeezing together of the handles 26, 28 to the disposition shown in FIG. 3 is terminated, the load spring 36 urges the handle 28 to move away from the handle 26, so as to return to the disposition shown in FIG. 1. FIG . 2 illustrates the disposition of the relevant components at an intermediate position between the disposition shown in FIG. 1 and the disposition shown in FIG. 3.

FIG. 4 illustrates, in solid lines, the disposition of the handles 26, 28, and the connecting pin 38 in the longitudinal recess 40 when in the disposition shown in FIG. 3, and in broken lines, when in the disposition shown in FIG. 1. For purposes of clarity, the flexible boot 37 is not shown in FIG. 4, as it forms no part of the essential operating structure of the present invention. As will be apparent from FIG. 4, the connecting pin 38 has moved upwardly in the longitudinal recess 40 in response to the arcuate movement of the handle 28 toward the handle 26, thereby enabling the connecting pin 38 to drive the piston rod 34, and so the piston 22, completely linearly in a longitudinal direction within the cylinder 20, as is shown in comparing FIGS. 1, 2 and 3. This completely linear longitudinal movement of the piston 22 is to be compared with the longitudinal movement of a corresponding piston in the prior art structures, which results in the canting of the piston and consequent scuffing of the O-ring seal, which necessarily results from the inability of its connecting pin to freely move vertically in order to compensate for the arcuate drive motion applied by the movable handle to the piston rod.

The invention, as illustrated in FIGS. 1 through 7, is shown as being implemented in a pump of the Class 2 lever type, i.e., as shown in U.S. Pat. No. 6,558,130. However, as will be apparent to those of ordinary skill in the art, the present invention is equally applicable to pumps of the Class 1 lever type, i.e., as shown in U.S. Pat. No. 4,775,302. Because of the difference in the fulcrum point of the handle in these two type pumps, the load spring is located outside of the cylinder in the Class 2 type pump, so as to retract the movable handle to its rest position, whereas in the Class 1 lever type pump, the load spring is located within the cylinder so as to retract the movable handle to its rest position as is shown in FIG. 1 of Pat. No. 4,775,302. Such a change in the positioning of the load spring is within the skill of those in the art, and so is within the scope of the present invention.

Although the presently preferred embodiments of the invention have been set forth herein in detail for illustrative purposes, it will be apparent to those skilled in the art that variations and modifications thereof, including the rearrangement of parts, lie within the scope of the present invention, which is not limited to the specific structures of the embodiments shown or described herein, but only by the scope of the following claims.

The invention claimed is:

1. In a hand held vacuum and pressure pump of the type which has
a fixed handle, with a movable handle pivotally connected thereto so as to be arcuately movable with respect thereto, the fixed handle being fixed to a pump cylinder so as to be perpendicular thereto, the pump cylinder containing a piston to which a piston rod is connected at one end, a load spring disposed along at least a portion of the piston rod, and valve means which controls whether the pump acts as a vacuum pump or a pressure pump, whereby the pump is operated by selective relative movement between the movable handle and the fixed handle, the improvement comprising:
a longitudinal recess formed in the movable handle;
a connecting pin extending through the longitudinal recess; and
means connecting the connecting pin to the piston rod at the piston rod end remote from the piston;
whereby, in response to arcuate movement of the movable handle toward and away from the cylinder during pump operation, the connecting pin moves longitudinally in the movable handle longitudinal recess, so that the piston rod moves linearly when moving longitudinally in the cylinder, thereby avoiding canting the piston in the cylinder during piston rod movement.

2. In a hand held vacuum and pressure pump of the type which has a fixed handle with a movable handle pivotally connected thereto at one end thereof, the fixed handle being fixed to a pump cylinder so as to be perpendicular thereto, a spring loaded piston rod which extends from the movable handle through the fixed handle and is attached at one end to a piston contained in the pump cylinder, the piston having an O-ring forming a seal between the piston and the cylinder, and valve means which selectively controls whether the pump acts as a vacuum pump or a pressure pump, and which is operated by manual movement of the movable handle toward the fixed handle, the improvement comprising:
a longitudinal recess formed in the movable handle;
a connecting pin longitudinally slidably disposed in the longitudinal recess; and
means connecting the connecting pin to the piston rod at the piston rod end remote from the piston;
whereby the arcuate movement of the movable handle toward and away from the cylinder permits the connecting pin to move longitudinally in the movable handle longitudinal recess, so that the piston rod moves linearly when moving longitudinally in the cylinder, thereby avoiding canting the piston during piston rod movement.
